(19) United States
(12) Patent Application Publication
(10) Pub. No.: US 2015/0276697 A1
(43) Pub. Date: Oct. 1, 2015

(54) Determining emission values

(71) Applicants: Klaus Hufschlag, Bad Honnef (DE); Patric Pütz, Andernach (DE); Ozan Sambur, Bonn (DE); Michael Wentzlaf, Bonn (DE); Andrea Sattler, Bonn (DE)

(72) Inventors: Klaus Hufschlag, Bad Honnef (DE); Patric Pütz, Andernach (DE); Ozan Sambur, Bonn (DE); Michael Wentzlaf, Bonn (DE); Andrea Sattler, Bonn (DE)

(73) Assignee: DEUTSCHE POST AG, Bonn (DE)

(21) Appl. No.: 14/665,572

(22) Filed: Mar. 23, 2015

(30) Foreign Application Priority Data
Mar. 28, 2014 (DE) 10 2014 104 361.8

(51) Int. Cl. G01N 33/00; G06Q 10/08

(52) U.S. Cl. CPC G01N 33/0036; G01N 33/0062; G06Q 10/0833

(57) ABSTRACT

An apparatus receives from a server emission-relevant data relating to transport operations on route sections. It determines a respective emission factor for a plurality of routes, which can be composed of the route sections, based on the emission-relevant data received. Each factor represents an emission value for a transport operation on the entire route per transport unit. The emission factors are stored with association with a starting location and a destination of the entire route. A request may be received relating to emission values regarding a specific conveying operation. It determines a starting point and a destination for the specific conveying operation and reads at least one stored factor associated with the starting location and destination. It determines a value for a transport unit associated with the factor, calculates an emission value from the factor and the determined numerical value for the transport unit, and transmits the emission value.

### CROSS-REFERENCE TO RELATED PATENT APPLICATIONS

This patent application claims priority to German Application No. 10 2014 104 361.8, filed on Mar. 28, 2014, the entire teachings and disclosure of which are incorporated herein by reference thereto.

### FIELD OF THE INVENTION

The present invention relates inter alia to a method, an apparatus, a system, a program and a storage medium for determining emission values, in particular greenhouse gas emission values.

### BACKGROUND OF THE INVENTION

Nowadays many companies, as a result of legal provisions, but also as a result of their own objectives, attempt to determine and where applicable reduce the emissions caused by them and their energy consumption. This also applies in particular to companies which offer conveying of goods or persons. The emissions taken into account may in particular comprise greenhouse-gas-related emissions, such as carbon dioxide (CO₂) and CO₂ equivalents. CO₂ equivalents, often shortened to CO₂e, are other greenhouse gases, such as methane (CH₄), nitrous oxide (N₂O), fluorocarbons, perfluorocarbons, sulphur hexafluoride (SF₆) and nitrogen trifluoride (NF₃) which are each standardized by means of a factor to CO₂ so that in each case 1 kg of CO₂ or CO₂ equivalent has the same greenhouse gas effect. Companies which provide conveying services therefore often collect emission-relevant data relating to the transport thereof.

In addition, private and commercial users of conveying services are also interested in environmentally relevant data relating to a specific conveying operation, such as, for instance, the individually caused quantity of greenhouse gases.

The emission values and the energy consumption for specifically conveying an item or a person may, for example, be individually established by means of a model calculation, assuming a probable route and average transport means. Ad hoc model calculations are a method which is currently commonplace.

Both for the emissions and for the energy consumption, it is possible to take into account, for example, only the effect of the actual transport operation (TTW: Tank-to-Wheel).

Alternatively or additionally, the effect resulting from the acquisition and provision of the energy carriers used for the transport operation can be taken into account (WTT: Well-to-Tank). Finally, the total of both can also be taken into account (WTW: Well-to-Wheel).

For the calculation and provision of energy consumption values and greenhouse gas emission values in the logistics sector there are various standards. The European Standard EN 16258 “Methology for calculation and declaration of energy consumption and GHG emissions of transport services (freight and passengers)” was formulated by the European Committee for Standardisation (CEN: Comité Européen de Normalisation). Another basis for the calculation and declaration of greenhouse gases is the French environmental protection law “Grenelle 2” (Regulation 2011-1336). EN 16258 makes provision for TTW and WTW values to be established and declared for all greenhouse gases and for the energy consumption. The regulation 2011-1336 in contrast makes provision only for WTW values for CO₂ emissions to be established and declared. The additional indication of corresponding TTW values is optional.

### SUMMARY OF THE INVENTION

An object of the invention is to make it possible to determine individual emission values for a multitude of conveying operations in a precise and rapid manner.

An exemplary first method according to the invention comprises, carried out by an apparatus, receiving emission-relevant data relating to a multitude of carried out transport operations on route sections from at least one server, the transport operations having been carried out within a closed time period. The method further comprises determining a respective emission factor for a plurality of possible entire routes which can be composed of the route sections, based on the emission-relevant data received, each emission factor representing an emission value for a transport operation on the entire route per transport unit. The method further comprises causing a storage of the determined emission factors for the plurality of possible entire routes in a memory with association with a starting location and a destination which define the respective entire route.

Complementary to this, an exemplary second method according to the invention, carried out by an apparatus, comprises receiving a request from a user device relating to emission values with respect to a specific conveying operation. The method further comprises determining a starting location and a destination for the specific conveying operation. The method further comprises reading at least one stored emission factor, which is associated with the determined starting location and destination, for an entire route from a memory, the at least one emission factor being based on emission-relevant data relating to a multitude of carried out transport operations on route sections, from which the entire route can be composed, in a closed period of time. The method further comprises determining a numerical value for a transport unit which is associated with the emission factor. The method further comprises calculating an emission value from the read emission factor and the determined numerical value for the transport unit and causing a transmission of the emission value to the user device.

The invention thus makes provision, for specific exemplary embodiments, for entire-route-specific emission factors to be extensively pre-calculated on the basis of actual emission values which are determined or calculated in a granular manner and stored. The actual emission values in this instance relate to values for carried out transport operations on route sections. Route sections may, for instance, be transport sections without a change of transport means, and a carried out transport operation may be the entire transport by a transport means on the respective route section. The entire route is intended to be understood to be the entire route on which an item or a person could be conveyed. The emission factors may set out an emission value per transport unit, that is to say, for example, per kilogram. If an indication relating to an emission value for a specific conveying operation of at least one item or at least one person on an entire route is desired, the emission value can be calculated from a stored emission factor for this entire route and a numerical value for the transport unit, that is to say, for example, the weight for an item which has been is transported or which is to be transported.

The invention is based on the consideration that model calculations cannot represent an actual routing and actually used transport means. In addition, a significant calculation time for determining a value for the respective entire route is required with each request, which may be problematic with a large number of shipments or other types of conveying operations.

One possible advantage of the invention is that, owing to taking into account a multitude of transport operations actually carried out over a respective time period, the precision of emission values which can be achieved for a specific conveying operation can be improved. Another possible advantage of the invention is that, as a result of the request-independent provision of emission factors for possible entire routes in a memory, the calculation time for the calculation of emission values for a specific conveying operation in response to a request can be considerably reduced.

Of course, the features of all the exemplary embodiments described in this document can be used in conjunction both with the first and with the second method according to the invention.

An exemplary apparatus according to the invention comprises means for carrying out any embodiment of the first method according to the invention and/or means for carrying out any embodiment of the second method according to the invention. The apparatus may, for example, be a server or a component for a server.

An exemplary apparatus according to the invention comprises at least one processor and at least one storage medium, with program instructions being stored in the at least one storage medium and the at least one storage medium and the program instructions being configured, with the at least one processor, to at least cause the apparatus to carry out the first and/or the second method according to the invention when the program instructions are carried out on the processor. The apparatus is, for example, configured in terms of software so as to be able to carry out the method. The term “configured in terms of software” is intended to be understood in particular to refer to the preparation of the apparatus which is required in order to be able to carry out a method, for example, in the form of program instructions on the processor.

A processor is intended to be understood, inter alia, to be one or more control units, microprocessors, microcontrol units, such as microcontrollers, digital signal processors (DSP), application-specific integrated circuits (ASIC) or Field Programmable Gate Arrays (FPGA).

A storage medium is, for example, a program memory and/or a main memory of the processor. The term “program memory” is intended to be understood to be inter alia a non-volatile memory and the term “main memory” is intended to be understood to be a volatile or non-volatile memory, in particular a memory having random access (RAM) and/or a flash memory. Non-volatile memories are, for example, memories having random access, such as, for example, NOR flash memories, or with sequential access, such as, for example, NAND flash memories, and/or memories having read only access (ROM), such as, for example, EPROM, EEPROM or ROM memories. The storage medium may, for example, be constructed in tangible form.

An exemplary system according to the invention comprises at least one apparatus according to the invention and at least one server which is configured to collect and provide data for a multitude of transport operations carried out on route sections.

Exemplary program instructions according to the invention cause an apparatus to carry out any embodiment of the first method according to the invention and/or any embodiment of the second method according to the invention when the program instructions are carried out by the processor. Program instructions can, for example, be distributed over a network, such as a local area network, a wide area network, a virtual network, a radio network, such as a mobile radio network, another telephone network and/or the Internet. Program instructions may be at least partially software and/or firmware of a processor. Program instructions according to the invention may, for example, be stored in a storage medium of an apparatus according to the invention. The program instructions may, for example, belong to one or more Java components. A plurality of Java components may communicate with each other by means of RMI (Remote Method Invocation). Of course, the program instructions may be part of one or more programs.

An exemplary storage medium according to the invention stores the program instructions according to the invention. The storage medium is, for example, a computer-readable storage medium which contains the program instructions according to the invention and which is, for example, constructed as a magnetic, electrical, electromagnetic, optical and/or other storage medium. The storage medium may in particular be a physical and/or tangible storage medium. The storage medium is, for example, portable or securely installed in an apparatus. The term “computer-readable” is intended to be understood to mean in particular that the storage medium can be read (out) and/or written by a computer or a data processing apparatus, for example, by a processor. The storage medium is, for example, a program memory of a processor.

In an exemplary embodiment of the invention, the emission-relevant data comprise at least one of the following parameters for a transport operation on a route section: an indication of a starting location, an indication of a destination, an indication of a CO₂ quantity, an indication of a CO₂ equivalent quantity, an indication of ton-kilometers, an indication of a nitrogen oxide quantity, an indication of a sulphur oxide quantity, an indication of a particulate matter quantity, an indication of a quantity of another type of emission, an indication of a distance travelled, an indication of a total weight of a transport means used, an indication of a loading weight of a transport means used, an indication of a loading volume of a transport means used, an indication of a loading capacity of a transport means used, an indication of a capacity utilization of a transport means used, an indication of a fuel consumption, an indication of a specific fuel consumption, an indication of a used fuel type, an indication of a type of transport means used, an indication of a flight number of an aircraft used, an indication of a number of passengers and/or an indication of a mean weight of passengers.

Of course, the emission-relevant data received may also have only a portion of these data, the emission-relevant data received may have different data for different transport operations, and the emission-relevant data received may also comprise additional data. Furthermore, in addition to emission-relevant data, other data may also be received, such as an indication of the report time period or the date of the transport on a respective route section.

Starting location and destination may be indicated, for example, in the form of geographical coordinates, in the form of location names, postal codes or complete addresses, or in the form of airport abbreviations or abbreviations for sea ports and inland ports. Fuel consumption and fuel type enable, together with predetermined factors, a calculation of different emission values. Possible fuel types may include, for example, diesel, kerosene, heavy oil and/or electrical power. An indication of a type of transport means may permit conclusions to be made relating to the type of fuel, if this has not been indicated. A type of transport means could, for example, be selected from the types aircraft, lorry and ship, etcetera; alternatively, however, more fine classifications could also be provided with different aircraft types, utility vehicle types and/or ship types. In addition to a conclusion with respect to the fuel used, a conclusion with respect to other parameters, such as the loading capacity, may also thereby be possible. A fuel consumption may be given in kilograms or liters. A specific fuel consumption may, for example, be given in liters per 100 km. CO₂ quantities, CO₂e quantities and other emission quantities may, for example, be given in kilograms. A distance travelled may be a distance which is usual for a route section, but optionally also comprise specifically taken diversions. A great circle distance (GCD) could also alternatively be used as the distance travelled. A loading weight may be indicated as a gross and/or net weight.

In an exemplary embodiment of the invention, the transport unit refers to the parameter weight. The transport unit could then be, for instance, kilograms or tons. In an exemplary embodiment, the transport unit refers to the parameter transport capacity. The transport unit could then be, for instance, ton-kilometers. In an exemplary embodiment, a transport unit refers to the parameter volume. The transport unit could then be, for instance, liters, cubic meters or containers. In an exemplary embodiment, a transport unit refers to a number of persons. Even in the case of personal transport, however, the transport unit could be a weight unit, such as kilograms, a mean weight per person being able to be assumed in this instance. In particular mixed transport forms are thereby able to be more readily evaluated.

In an exemplary embodiment of the invention, a respective emission value refers to WTW emissions, TTW emissions or WTT emissions. In an exemplary embodiment, a respective emission value refers to CO₂ emissions, to CO₂ equivalent emissions, to nitrogen oxide emissions, to sulphur oxide emissions or to particulate matter emissions. The emission value may in this instance refer to both the emission value per transport unit which forms the basis of the emission factor and the emission value provided for a user device. Such emission values can be used to be able to correspond to at least one specific standard. In this instance, it should be noted that the third value can be determined in each case from two of the values WTW, TTW and WTT, without a separate emission factor having to be stored for this purpose.

In an exemplary embodiment of the invention, there is further determined a respective energy consumption factor for the plurality of possible entire routes, based on the emission-relevant data received, each energy consumption factor representing an energy consumption for a transport operation on the entire route per transport unit. In this instance, a storage of the energy consumption factors determined for the plurality of possible entire routes in the memory may also be caused, with association with the starting location and destination which define the respective entire route. Such additional energy consumption factors may be used in order to be able to comply with specific standards, such as EN 16258. In a similar manner to the emissions, the energy consumption may refer to a WTW energy consumption (and consequently to a primary energy consumption), a TTW energy consumption (and consequently to a final energy consumption) or a WTT energy consumption.

Of course, the term energy consumption is intended to be understood in the colloquial and economic sense and is intended to refer to the energy requirement for a specific application (for example, diesel consumption in liters), the energy requirement determined in each case being able to be converted in exemplary embodiments into a standardized energy requirement in megajoules. The term energy consumption is nonetheless used continually here since it is also used, for example, in EN 16258.

For each entire route, only one emission factor could be determined and stored. However, it would also be possible to determine and store a plurality of emission factors for a plurality of emission values. In an exemplary embodiment, the determination of a respective emission factor for the plurality of possible entire routes comprises the determination of at least two respective emission factors for the plurality of possible entire routes. The at least two respective emission factors can then be determined in accordance with at least two different standards, wherein causing a storage of the determined emission factors may comprise causing a separate storage of the emission factors determined in accordance with different standards. It would thus, for instance, be possible to store the emission factors for different standards in different tables. The different standards may comprise, for example, EN 16258, the French regulation 2011-1336 or any other existing or future standard.

In an exemplary embodiment of the invention, emission-relevant data are received from the at least one server at predetermined intervals, and the determination of a respective emission factor for a plurality of possible entire routes is based on the emission-relevant data last received in each case. The data may, for example, in each case relate to transport operations within a closed time period of predetermined length.

Over time, improved technologies may lead to reduced fuel consumption. Furthermore, over time, new route sections for transport operations may be incorporated. Such effects may be taken into account by the regular recalculation of emission factors. For the emission factors, two different memories or at least two different database instances which use the same memory could be provided. This could have the advantage that the old emission factors can be provided in each case without impairment, until the calculation of the new emission factors is fully complete.

In an exemplary embodiment of the invention, the determination of emission factors comprises a calculation of at least one emission value per transport unit for a carried out transport operation on a respective route section based on received emission-relevant values for the carried out transport operation. The emission-relevant data could thus comprise, for example, an emission value for a carried out transport operation on a route section and the freight weight of the transport. The emission value per transport unit for the carried out transport operation could then be determined by dividing the emission value received by the freight weight.

In an exemplary embodiment, the determination of emission factors alternatively or additionally comprises a calculation of at least one emission value per transport unit for a respective carried out transport operation on a respective route section based on received emission-relevant values for the carried out transport operation and based on stored provisions. Such provisions could, for example, comprise conversion factors with which an indication of a used quantity of a specific fuel type can be converted into one or more emission values. The provisions could further comprise, for example, conversion factors, with which an emission value can be converted to another emission factor or an energy consumption factor in a manner specific to the fuel type. The provisions could further comprise, for example, an association of transport means types with fuel types in case that, in the emission-relevant data received, only the type of transport means but not the fuel type is indicated. The provisions could further, for example, comprise the empty weight of different transport means types in case that only a total weight is transmitted with the emission-relevant data.

In an exemplary embodiment, the determination of emission factors alternatively or additionally comprises generating a mean value of emission values per transport unit for a respective route section received or calculated for a plurality of carried out transport operations on the respective route section. A representative value for each route section is thereby produced. The mean value may in this instance be any mean value, such as an arithmetic mean value, a quadratic mean value or a median.

In an exemplary embodiment, the determination of emission factors alternatively or additionally comprises a totaling of emission values per transport unit for different route sections which form a respective entire route. The totaled emission values may in this instance, for example, be emission values which are averaged per route section.

In an exemplary embodiment of the invention, at least a portion of the emission factors is based on fuel data relating to carried out transport operations, that is to say, on information relating to actual fuel consumption. This could be based, for example, on fuel bills or fuel card information. The fuel data may either be received from the server and used in the apparatus for determining the emission factors, or they can already be used by the server for calculating intermediate values, which are then transmitted to the apparatus and are further used at that location for determining the emission factors. Another portion of the emission factors may be based on operating data and/or economic data.

In an exemplary embodiment of the invention, after the storage of determined emission factors in the memory, a request is received from a user device relating to emission values with respect to a specific conveying operation, a starting location and a destination are determined for the specific conveying operation, at least one stored emission factor, which is associated with the determined starting location and destination, for an entire route is read from the memory, a numerical value is determined for a transport unit which is associated with the emission factor, an emission value is calculated from the read emission factor and the determined numerical value for the transport unit, and a transmission of the emission value to the user device is caused.

In an exemplary embodiment of the invention, such a request from a user device is enabled in the context of a service which enables a shipment monitoring (Track and Trace Service). In an exemplary embodiment of the invention, the request may be carried out via the interception-proof communication protocol HTTPS (HyperText Transfer Protocol Secure). However, it is self-evident that, for specific application fields, a non-secure connection may also be used. In an exemplary embodiment of the invention, the transmission of the emission value to the user device may be carried out in XML format (Extensible Markup Language). However, any other formats, such as HTML (Hypertext Markup Language) can also be used.

The request can specify the specific conveying operation in different manners. If the specific conveying operation relates to a shipment which has already been passed to a logistics company for conveying, it can be specified, for example, by means of an identification (shipment ID) which is associated with the shipment. If the specific conveying operation refers to a shipment which has not (yet) been submitted, it may be specified, for example, by indication of a starting location, a destination, and a weight. If the specific conveying operation relates to conveying of persons, it may be specified, for example, by indication of a starting location, a destination and a number of persons.

An exemplary embodiment of the invention comprises the provision of a web service, which enables the request via the user device. In an exemplary embodiment, the web service may, for example, be a web service for tracking a shipment. Another exemplary embodiment of the invention comprises the provision of a web service for tracking a shipment using a user device which supports the request via the user device, wherein the specific conveying operation is indicated by means of a shipment identification which is input via the user device, and wherein the starting location and the destination for the specific conveying operation and the numerical value for the transport unit for the specific conveying operation are determined by means of the shipment identification. Another exemplary embodiment of the invention comprises the provision of a web service, which enables the request via the user device and which enables an input of the starting location and the destination for the specific conveying operation and the numerical value for the transport unit for the specific conveying operation via the user device.

In an exemplary embodiment of the invention, the request comprises an indication of at least one desired standard, wherein the reading of at least one emission factor from the memory comprises reading at least one emission factor which is stored for the at least one desired standard.

Other advantageous exemplary embodiments of the invention can be taken from the following detailed description of a few exemplary embodiments of the present invention, in particular together with the figures. However, the Figures are intended only for purposes of illustration, but not to determine the scope of protection of the invention. The figures are not true to scale and are intended to reflect only the general concept of the present invention by way of example. In particular, features which are contained in the figures are not intended in any way to be considered to be an absolutely necessary component of the present invention.

### BRIEF DESCRIPTION OF THE DRAWINGS

In the drawings:

FIG. 1 is a block diagram of an exemplary embodiment of an apparatus according to the invention in an exemplary embodiment of a system according to the invention.

FIG. 2 is a flow chart with method steps of an exemplary embodiment of a first method according to the invention.

FIG. 3 is a flow chart with method steps of an exemplary embodiment of a second method according to the invention.

FIG. 4 is a schematic illustration of first exemplary emission information.

FIG. 5 is a schematic illustration of other exemplary emission information.

### DETAILED DESCRIPTION OF THE INVENTION

The present invention is described below with reference to exemplary embodiments.

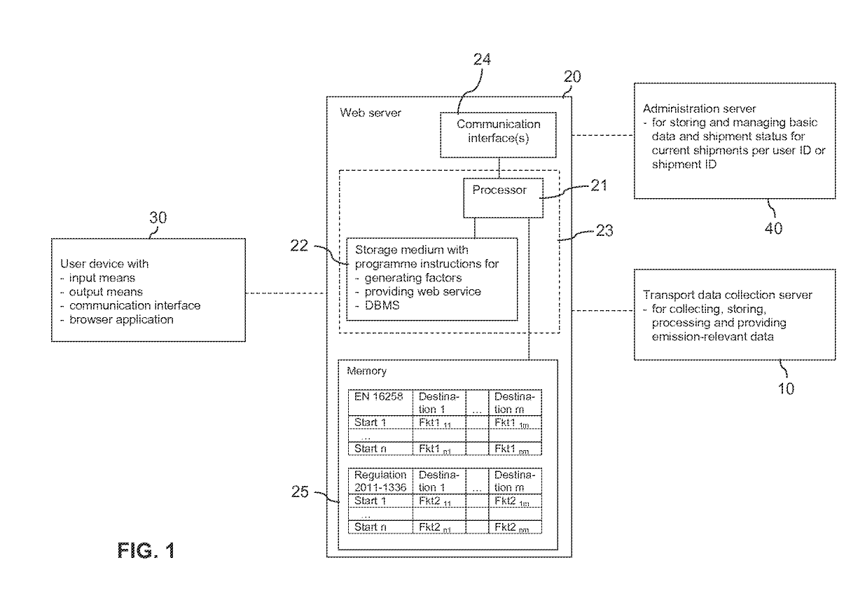

FIG. 1 is a block diagram of an exemplary embodiment of a system according to the invention.

The system comprises a transport data collection server 10, a web server 20, a user device 30 and an administration server 40.

The transport data collection server 10, the web server 20 and the administration server 40 could, for example, belong to a logistics company which transports parcels or goods for its customers and which would like to provide information to the customers relating to greenhouse gas emissions caused by the individual shipments.

The transport data collection server 10 is configured to collect, to store, to process and to provide extensive data relating to transport operations on route sections.

The web server 20 comprises, for example, a processor 21 and, connected to the processor 21, a storage medium 22. The storage medium 22 stores program instructions which may belong, for example, to different Java components. The processor 21 is configured to carry out program instructions from the storage medium 22 and thereby to cause the web server 20 to carry out specific actions. The program instructions comprise program instructions for generating emission and energy consumption factors and program instructions for providing a web service for users. The program instructions could also comprise program instructions for a database management system (DBMS), for example, for a PostgreSQL database which the Java components can access. In the storage medium 22, it would further be possible to store data, such as, for example, predetermined parameters, which are accessed when the program instructions are carried out. Furthermore, any other types of data could also be stored in the storage medium 22. The processor could be a microprocessor. It could also be embedded in an integrated circuit (IC) 23, for example, together with the storage medium 22. Component 23 could instead, for example, also be a plug type card with processor 21 and storage medium 22.

The web server 20 further comprises, by way of example, at least one communication interface 24. The communication interface 24 is connected to the processor 21 and enables a data exchange with other external devices, such as, for example, the transport data collection server 10, the user device 30 and the administration server 40, via the Internet. If a plurality of communication interfaces is provided, another data exchange could take place via other communication channels, for example, via a local area network. Such a local connection could, for example, be established to one or both of the other servers 10, 40 when they are arranged on the same operating premises.

The web server 20 further comprises, for example, a memory 25 for a plurality of database instances of a database managed by the database management system. The memory 25 is also connected to the processor 21. The memory 25 could optionally also be arranged separately from the web server 20. In this case, the communication between the web server 20 and the memory 25 could also take place via the at least one communication interface 24.

The web server 20 may optionally comprise any other components which are not illustrated, for example, additional processors or stores, in particular also an operating memory for the processor 21.

Web server 20 or component 23 are exemplary embodiments of an apparatus according to the invention. In another exemplary embodiment of an apparatus according to the invention, the apparatus could comprise only a circuit in which corresponding functions are implemented in terms of hardware.

The user device 30 could, for example, be a PC, a laptop, a tablet computer, a smartphone or another apparatus. The user device 30 comprises input means, such as, for example, a keyboard or a touch screen, and output means, such as, for example, a display. The user device 30 further comprises a communication interface, which enables an Internet connection and a browser application which enables a user to access the Internet and web services.

The administration server 40 is configured to collect, to store and to administer basic data relating to shipments per shipment ID and/or user ID, and to provide and continuously update a shipment status for each shipment. To this end, it can receive information from various entities of the logistics company, such as, for example, parcel receipt locations, distribution centers or delivery drivers.

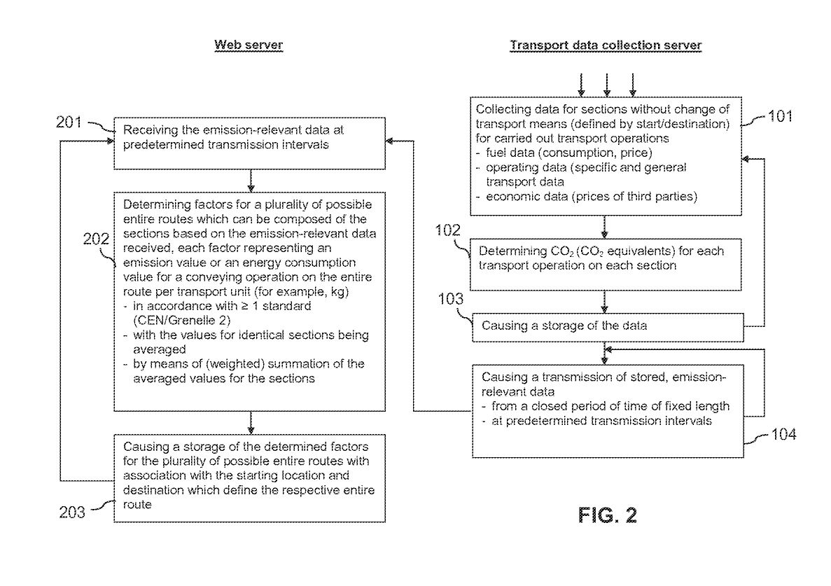

FIG. 2 illustrates a first operation in the system from FIG. 1 according to an exemplary embodiment of the first method according to the invention. The actions shown at the right-hand side are carried out by the transport data collection server. The actions shown at the left-hand side are carried out by the web server 20 when the processor 21 carries out the program instructions for generating emission and energy consumption factors from the storage medium 22.

The server 10 collects constantly or at regular intervals data relating to carried out transport operations on route sections without change of transport means. (Action 101) The transport operations may, for example, comprise all transport operations which are carried out or utilized by the logistics company. A change of transport means may be a change between different types of transport means, that is to say, for example, a change from a utility vehicle, such as a lorry, to an aircraft, or a change between transport means of the same type, that is to say, for example, a change from one aircraft to another.

The data collected may, for example, comprise fuel data, operating data and economic data.

The server 10 determines from the collected data and from stored parameters per transport operation on each route section the quantity of CO₂ emissions and/or CO₂e emissions caused. (Action 102) Stored parameters may, for example, comprise various emission factors, such as CO₂ in kg per consumed kilogram or liter of fuel per fuel type and/or transport means type, the mean fuel costs per fuel type, the percentage proportion of fuel costs of the overall transport costs, etcetera.

The emissions can be calculated from the fuel consumption, which can be produced from invoices or the information from fuel card administration systems. If only the fuel costs are known, the fuel consumption can be estimated on the basis of the mean fuel costs for a specific fuel type.

The emissions may alternatively be estimated from operating data. For flights, this may include the actual or estimated freight weight, the maximum freight in kg and a percentage capacity utilization, in addition optionally the aircraft type, with each aircraft type being able to be associated with a specific emission factor. For transport operations on the road, the distances travelled per vehicle class, or a calculated mean distance per vehicle and vehicle class, the number of vehicles and a mean fuel consumption per vehicle class could be taken into account. Alternatively, ton-kilometers per vehicle class can be taken into account. For transport operations by ship, the distance travelled and the TEU (Twenty-foot Equivalent Unit) per harbor pair can be taken into account.

The emissions can further alternatively be estimated from economic data. These may, for example, comprise the costs for transport operations by third parties, wherein a specific percentage can be assumed for the fuel costs contained therein. The fuel consumption can in turn be determined therefrom.

As long as no other data are available, the emissions can further alternatively be estimated by models.

Of course, the data and parameters set out are merely examples.

The server 10 causes for each transport operation on each route section a storage of the data received, the emission values determined and where applicable other calculated data. (Action 103) In this way, several million sets of data, with a volume of several hundred megabytes, may be generated and stored by the server 10 per month and transport means.

At predetermined intervals, for example, once a quarter, the server 10 causes a transmission of stored emission-relevant data per transport operation on a route section for a closed period of time of fixed length, for example, for the last half of the year, to the web server 20. (Action 104) Of course, not all stored data available to the server 10 have to be transmitted. The intervals, at which data are transmitted, are also referred to below as transmission intervals, and the respective period of time of fixed length, for which data are transmitted, is also referred to below as a report time period.

The web server 20 receives the emission-relevant data for a report time period at the predetermined transmission intervals. (Action 201)

The data received comprise for a respective transport operation on a respective route section, for example, an indication of a starting location of the route section, an indication of a destination of the route section, an indication of a CO₂ quantity and/or an indication of a CO₂e quantity. Additional indications for the transport operation may depend on the transport means used and the information available. These include, for example, an indication of ton-kilometers, an indication relating to the distance travelled, an indication relating to the overall weight of a transport means used, an indication relating to the loading weight of a transport means used, an indication relating to the loading volume of a transport means used, an indication of the loading capacity of a transport means used, an indication of the capacity utilization of a transport means used, an indication relating to the fuel consumption, an indication relating to a fuel type used, an indication relating to a type of transport means used, an indication relating to the flight number of an aircraft used, an indication relating to a flight duration, an indication relating to a number of passengers involved in the transport operation, and an indication relating to a mean weight of passengers involved in the transport operation.

The web server 20 now determines, based on the data received for the route sections, emission factors for entire routes, each emission factor representing an emission value per transport means unit, for instance, per kilogram. (Action 202)

The specific entire route for conveying a specific item can be composed of route sections.

Each of the route sections has a starting location and a destination, a large number of data sets potentially being available for a route section since most route sections are travelled frequently within a report time period. The web server 20 therefore first combines the data for the transport operations on a respective route section.

For example, the web server 20 can first determine for each carried out transport operation on a respective route section based on the emission-relevant data received, a TTW CO₂e emission factor, a WTW CO₂e emission factor, a TTW energy consumption factor, a WTW energy consumption factor, a TTW CO₂ emission factor or a WTW CO₂ emission factor. Each of the factors represents an emission value or an energy consumption value per transport unit, that is to say, for example, a quantity of CO₂ in kilograms per kilogram of freight or persons. If, for each transport operation on a route section, a TTW CO₂e quantity is transmitted by the server 10, the web server 20 could first determine a TTW CO₂e emission factor for each transport operation by dividing the TTW CO₂e quantity by the entire useful weight.

The relationship of the factors to each other is dependent on the type of fuel used for the transport operation, such as diesel, kerosene, heavy oil or electrical power. If one of the mentioned emission and energy consumption factors (with the exception of the TTW emissions in the case of electrical power as the fuel used) and an indication relating to the fuel type are determined, the other factors can be calculated therefrom by means of conversion factors stored in the web server 20 for the respective fuel type. If the data transmitted for a transport operation contain an indication of a type of transport means instead of an indication relating to the fuel type, the fuel type could be determined therefrom by means of an association stored in the web server 20.

For all route sections, the factors for all carried out transport operations on the same route section are then averaged so that an averaged factor for each transport route section is available.

The specific entire route for conveying a specific delivery may be composed of route sections.

The web server 20 could now determine, from the averaged factors for the route sections, for all possible entire routes a respective factor so that these entire route factors are immediately available when required. That is to say, each provided starting location of a route section would be combined with each provided destination of a route section in order to receive all possible entire routes. The averaged factors for the route sections, from which an entire route can be composed, could then be totaled in order to receive the entire route factor. In order to limit the required calculation capacity and the necessary storage requirement, however, the web server 20 in the present embodiment determines only entire route factors for all entire routes which have actually occurred or all probable entire routes. In this instance, the averaged factors for the route sections, from which a respective entire route can be composed, is also totaled in order to receive the entire route factor. Information relating to entire routes which have occurred to date or in the last report time period could, for example, be provided by the transport data collection server 10 or by the administration server 40. Probable entire routes could be determined, for example, from the end locations of entire routes which have occurred.

The web server 20 causes a storage of the factors for the entire routes in the memory 25. (Action 203) The factors may, for example, be stored as elements of tables. As indicated in FIG. 1 with regard to the memory 25, a table can associate at least one factor Fkt1₁₁ to Fkt1ₙₘ or a factor Fkt2₁₁ to Fkt2ₙₘ with each combination of a starting location 1 to n and a destination 1 to m.

In this instance, it is possible, for example, to compose a first table for factors according to EN 16258 and a second table for factors according to the French regulation 2011-1336. In the table for EN 16258 there can be associated with each starting location/destination pair a TTW CO₂e emission factor, a WTW CO₂e emission factor, a TTW energy consumption factor and a WTW energy consumption factor as a table element. In the table for the regulation 2011-1336, a TTW CO₂ emission factor and a WTW CO₂ emission factor may be associated with each starting location/destination pair as a table element.

Of course, a further splitting of the tables would be possible. For example, a table for transport operations with an air transport portion and a table for transport operations without any air transport portion could be produced in each case. In this instance, for a respective route section, separately averaged factors for air transport operations and non-air-transport operations could be determined, with only the data sets for air transport operations or only the data sets for other transport operations, respectively, then being taken into account for the route section when the factors are determined. For the entire routes, where possible, two sets of entire route factors could then be determined, one for conveying operations with air transport and one for conveying operations without any air transport.

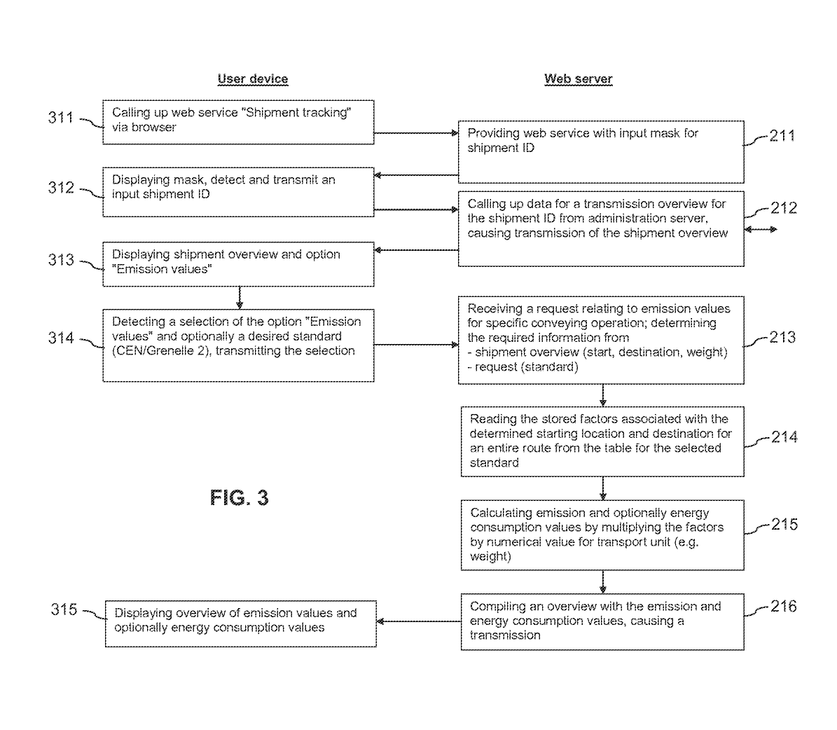

FIG. 3 illustrates a second operation in the system from FIG. 1 according to an exemplary embodiment of the second method according to the invention. The actions shown at the right-hand side are carried out by the web server 20 when the processor 21 carries out the program instructions for providing the web service from the storage medium 22. The actions shown at the left-hand side are carried out by the user device 30.

The web server 20 provides a web service of the logistics company for a shipment tracking. For use of the web service by means of a user device 30, it is possible to provide, for example, HTTPS. For illustrations provided by the web service, it is possible to provide, for example, the XML format.

When a user wishes to receive information relating to a shipment which is being handled by the logistics company, he can call up the web service “Shipment tracking” via a browser. (Action 311)

After the service has been called up, the web server 20 provides the user device with a webpage comprising an input mask in which, for example, a shipment ID which identifies a specific shipment can be input. (Action 211)

The user device 30 displays the input mask in the browser. The user can now, for example, input here a shipment identification for conveyance of a shipment which he has requested from the logistics company, in order to request a shipment overview. The input is registered by the user device 30 and the corresponding data transmitted to the web server 20. (Action 312)

The web server 20 receives the request and calls up current data relating to the shipment identified by the shipment ID from the administration server 40. The data can already be provided by the administration server 40 as a shipment overview in a suitable format. However, the shipment overview may also be produced only by the web server 20 based on the data received. In the latter case, the shipment overview may comprise all data received relating to the shipment ID or only selected data. The web server 20 then causes a transmission of a shipment overview to the user device 30 (Action 212).

The user device 30 receives the shipment overview and displays it in the browser. (Action 313) The overview may contain, for example, data such as the starting location, destination, weight and any other data relating to the shipment. If it is a collective shipment with a plurality of individual items, which are identified by means of a single shipment ID, it would be, for example, possible to also indicate the number of items. A collective shipment could, for example, be a container with a large number of items. The current status of the shipment may be part of the shipment overview or be transmitted separately via a separate request. A selectable option “Emission values” is further displayed to the user.

If the user selects this option, it is detected by the user device 30. Optionally, the user may at the same time or in a second step after selecting the option “Emission values” still select a desired standard for the indication of the emission values. Possible selections could include, for example, EN 16258 and/or the French regulation 2011-1336. However, it is self-evident that any other standards could additionally or alternatively be offered. It is further self-evident that a determination according to one or more standards can be fixedly predetermined so that a request is superfluous. The user device 30 transmits the request for the emission values and optionally an indication of the selected standard to the web server 20. (Action 314)

The web server 20 receives the request relating to the emission values for the specific conveying operation identified by the shipment ID and subsequently determines the information required for determining the emission values. (Action 213)

The web server 20 can thus derive from the shipment overview received from the administration server the starting location, the destination and the weight of the shipment. From the request transmitted by the user device 30, the web server 20 can, for example, derive an indication relating to an optionally selected standard.

The web server 20 now reads from at least one table in the memory 25 factors for the entire route, on which the identified shipment is conveyed. The relevant table or the relevant tables is/are determined according to the selected standard or the selected standards. From the respective table, the factors which are associated with the combination of the starting location and destination determined in action 213 are selected. (Action 214)

Each of the extracted factors is now multiplied by a numerical value for the transport unit, that is to say, in the present case multiplied by the determined weight in kg of the shipment in Action 213 in order to receive emission values or energy consumption values for the shipment. (Action 215)

As described in connection with Action 203, the tables could comprise TTW and WTW factors so that with the multiplication corresponding TTW and WTW values for the shipment on the entire route are produced. If necessary, for example, it is further possible to calculate WTT values from the TTW values and WTW values by a TTW value being subtracted from the corresponding WTW value in each case.

The web server 20 compiles an overview from the determined emission and optionally energy consumption values and causes a transmission to the user device 30. (Action 216)

The user device 30 receives the overview and presents it to the user in the browser. (Action 315)

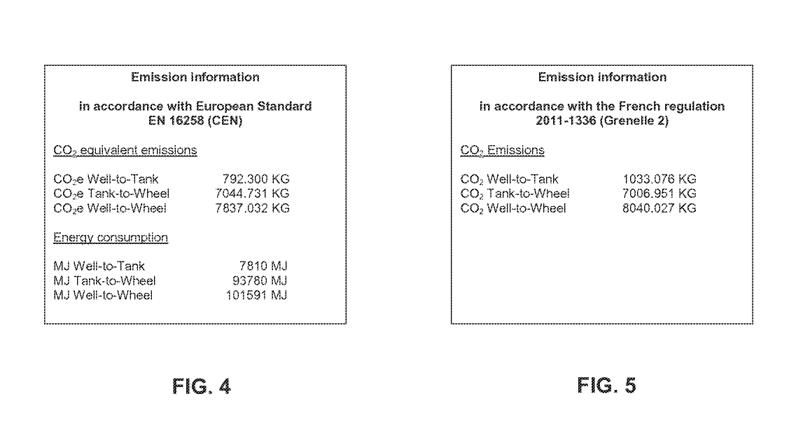

An exemplary overview of requested emission information in accordance with the European Standard EN 16258 is illustrated in FIG. 4. The illustration comprises exemplary values of CO₂ equivalent emissions CO₂e WTT, CO₂e TTW and CO₂e WTW and exemplary values of the energy consumption in megajoule MJ WTW, MJ TTW and MJ WTW.

An exemplary overview of requested emission information in accordance with the French regulation 2011-1336 is illustrated in FIG. 5. The illustration comprises exemplary values of CO₂ emissions CO₂ WTT, CO₂e TTW and CO₂ WTW.

If emission values in accordance with both standards have been requested, the overviews from FIG. 4 and FIG. 5 can be displayed by the user device 30 together in the browser.

Since the factors for the entire route of the shipment were previously pre-calculated, the overview can be displayed in the shortest possible time after the request for the emission values. Since the pre-calculated factors are based on detailed information relating to a large number of transport operations actually carried out on route sections, the values indicated may further be particularly realistic.

The system from FIG. 1 can be modified in varied ways, both by the omission of components and by the addition of components. Additional or alternative embodiments can thereby be obtained.

The methods from FIGS. 2 and 3 can also be modified in varied ways.

It would thus be possible, for example, for the functions of the administration server 40 also to be taken over by the web server 20.

Furthermore, it would be, for example, possible to provide a separate processor and separate program store for the database management system. The processor 21 could then access the memory 25 via this additional processor.

It would be further possible, for example, to provide a first memory 25 for retrieving determined factors in Action 214, and a second memory for storing the factors, whilst the factors are determined in Action 202. If the factors for a report time period are completely stored in the second memory, the data can be copied within a relatively short period of time into the first memory 25. It can thereby be ensured that, in the event of a recalculation of the factors, no relatively long interruption of the access to the web service is required. In another embodiment, it would be possible to store new factors alternately in the first and second memory and then to alternately read them from the memory in which an update was last completed.

Furthermore, it would be possible, for example, for the transport data collection server 10 to already carry out more extensive calculations and to provide them to the web server 20. On the other hand, it is not absolutely necessary for the transport data collection server 10 to determine emission values for the carried out transport operations and to transmit them to the web server 20. The emission values for the carried out transport operations could also be determined by the web server 20 on the basis of the remaining emission-relevant data received.

If separate factors for entire routes involving air transport and for entire routes not involving air transport are present in the memory 25, this can be taken into account in the selection of the factors. The data called up in the Action 212 for the shipment overview may thus also contain an indication as to whether or not air transport is planned for the shipment. This could then be taken into account when reading the factors in Action 214. It would thereby be possible to achieve an even higher level of precision when indicating the emissions caused and the energy consumed for a specific shipment.

The possibility of requesting emission values further does not have to be limited to actually ordered shipments. A user could be interested beforehand in which emissions are produced by a planned shipment. In this instance, an alternative input interface could be provided. When this is selected and displayed, the user can input delivery-relevant indications, such as starting location, destination and weight of the delivery. In Action 213, the web server 20 then determines these data not from data received from the administration server 40, but instead based on the user inputs.

A provision of pre-calculated factors for entire routes is further not limited to the conveying of goods. It could also be provided in a similar manner for the transport of persons. In this case, the transport unit could also be kilograms or a unit for the number of persons.

Of course, the pre-calculated factors can be used not only in the context of a web service for determining emission values and energy consumption values relating to individual conveying operations. They could also be used, for instance, for the determination of emission values and energy consumption values in the context of emission reports and statistics which a logistics company can provide for large customers.

The blocks illustrated in FIGS. 2 and 3 can also be understood as illustrations of means with the corresponding functions.

The connections illustrated or described between components are intended to be understood to be functional connections. They can be formed directly or indirectly via a plurality of other components. The sequence of the actions described in the individual flow charts is not compulsory; alternative sequences of the method steps are conceivable. The actions can be implemented in various manners. An implementation is thus conceivable not only in software (via program instructions) but also only in hardware or in a combination of both.

It is self-evident that the embodiments described are merely examples which can be modified and/or supplemented in varied ways within the scope of the claims. In particular, each feature which has been described for a specific embodiment can be used independently or in combination with other features in any other embodiment. Each feature which has been described for an embodiment of a specific category can also accordingly be used in an embodiment of another category.

1. A method carried out by an apparatus, the method comprising:
receiving emission-relevant data relating to a multitude of carried out transport operations on route sections from at least one server, the transport operations having been carried out within a closed time period of fixed length,
determining a respective emission factor for a plurality of possible entire routes which can be composed of the route sections, based on the emission-relevant data received, each emission factor representing an emission value for a transport operation on the entire route per transport unit, and
causing a storage of the determined emission factors for the plurality of possible entire routes in a memory with association with a starting location and a destination which define the respective entire route.

2. The method according to claim 1, wherein the emission-relevant data comprise at least one of the following parameters for a transport operation on a route section:
an indication of a starting location of the route section;
an indication of a destination of the route section;
an indication of a CO₂ quantity;
an indication of a CO₂ equivalents quantity;
an indication of a nitrogen oxide quantity;
an indication of a sulphur oxide quantity;
an indication of a particulate matter quantity;
an indication of ton-kilometers;
an indication of a distance travelled;
an indication of a total weight of a transport means used;
an indication of a loading weight of a transport means used;
an indication of a loading volume of a transport means used;
an indication of a loading capacity of a transport means used;
an indication of a capacity utilization of a transport means used;
an indication of a fuel consumption;
an indication of a specific fuel consumption;
an indication of a used fuel type;
an indication of a type of transport means used;
an indication of a flight number of an aircraft used;
an indication of a number of passengers; and
an indication of a mean weight of passengers.

3. The method according to claim 1, wherein a transport unit refers to one of the following parameters:
a weight;
a traffic capacity;
a volume; and
a number of persons.

4. The method according to claim 1, wherein an emission value refers to at least one of the following emissions:
Well-to-Wheel emissions;
Tank-to-Wheel emissions;
Well-to-Tank emissions;
CO₂ emissions;
CO₂ equivalents emissions;
nitrogen oxide emissions;
sulphur oxide emissions; and
particulate matter emissions.

5. The method according to claim 1, comprising:
determining a respective energy consumption factor for the plurality of possible entire routes based on the emission-relevant data received, each energy consumption factor representing an energy consumption for a transport operation on the entire route per transport unit; and
causing a storage of the determined energy consumption factors for the plurality of possible entire routes in the memory, with association with the starting location and the destination which define the respective entire route.

6. The method according to claim 1, wherein determining a respective emission factor for the plurality of possible entire routes comprises determining at least two respective emission factors for the plurality of possible entire routes, wherein the at least two respective emission factors are determined in accordance with at least two different standards, and wherein causing a storage of the determined emission factors comprises causing a separate storage of the emission factors determined in accordance with different standards.

7. The method according to claim 1, further comprising:
receiving a request from a user device relating to emission values with respect to a specific conveying operation;
determining a starting location and a destination for the specific conveying operation;
reading at least one stored emission factor, which is associated with the determined starting location and destination, for an entire route from the memory;
determining a numerical value for a transport unit which is associated with the emission factor;
calculating an emission value from the read emission factor and the determined numerical value for the transport unit; and
causing a transmission of the emission value to the user device.

8. The method according to claim 7, further comprising at least one of the following:
providing a web service which enables the request via the user device;
providing a web service for a shipment tracking by means of a user device which enables the request via the user device;
providing a web service for a shipment tracking by means of a user device which supports the request via the user device, wherein the specific conveying operation is indicated by means of a shipment identification which is input via the user device and wherein the starting location and the destination for the specific conveying operation and the numerical value for the transport unit for the specific conveying operation are determined by means of the shipment identification; and
providing a web service which enables the request via the user device and which enables an input of the starting location and the destination for the specific conveying operation and the numerical value for the transport unit for the specific conveying operation via the user device.

9. The method according to claim 7, wherein the request comprises an indication of at least one desired standard, and wherein the reading of at least one emission factor from the store comprises reading at least one emission factor which is stored for the at least one desired standard.

10. An apparatus comprising at least one processor and at least one storage medium, with program instructions being stored in the at least one storage medium and with the program instructions being configured to cause the apparatus to perform the following when executed by the at least one processor:
receive emission-relevant data relating to a multitude of carried out transport operations on route sections from at least one server, the transport operations having been carried out within a closed time period of fixed length,
determine a respective emission factor for a plurality of possible entire routes which can be composed of the route sections, based on the emission-relevant data received, each emission factor representing an emission value for a transport operation on the entire route per transport unit, and
cause a storage of the determined emission factors for the plurality of possible entire routes in a memory with association with a starting location and a destination which define the respective entire route.

11. The apparatus according to claim 10, wherein the emission-relevant data comprise at least one of the following parameters for a transport operation on a route section:
an indication of a starting location of the route section;
an indication of a destination of the route section;
an indication of a CO₂ quantity;
an indication of a CO₂ equivalents quantity;
an indication of a nitrogen oxide quantity;
an indication of a sulphur oxide quantity;
an indication of a particulate matter quantity;
an indication of ton-kilometers;
an indication of a distance travelled;
an indication of a total weight of a transport means used;
an indication of a loading weight of a transport means used;
an indication of a loading volume of a transport means used;
an indication of a loading capacity of a transport means used;
an indication of a capacity utilization of a transport means used;
an indication of a fuel consumption;
an indication of a specific fuel consumption;
an indication of a used fuel type;
an indication of a type of transport means used;
an indication of a flight number of an aircraft used;
an indication of a number of passengers; and
an indication of a mean weight of passengers.

12. The apparatus according to claim 10, wherein a transport unit refers to one of the following parameters:
a weight;
a traffic capacity;
a volume; and
a number of persons.

13. The apparatus according to claim 10, wherein an emission value refers to at least one of the following emissions:
Well-to-Wheel emissions;
Tank-to-Wheel emissions;
Well-to-Tank emissions;
CO₂ emissions;
CO₂ equivalents emissions;
nitrogen oxide emissions;
sulphur oxide emissions; and
particulate matter emissions.

14. The apparatus according to claim 10, wherein the program instructions are further configured to cause the apparatus to perform the following when executed by the at least one processor:
determine a respective energy consumption factor for the plurality of possible entire routes based on the emission-relevant data received, each energy consumption factor representing an energy consumption for a transport operation on the entire route per transport unit; and
cause a storage of the determined energy consumption factors for the plurality of possible entire routes in the memory, with association with the starting location and the destination which define the respective entire route.

15. The apparatus according to claim 10, wherein determining a respective emission factor for the plurality of possible entire routes comprises determining at least two respective emission factors for the plurality of possible entire routes, wherein the at least two respective emission factors are determined in accordance with at least two different standards, and wherein causing a storage of the determined emission factors comprises causing a separate storage of the emission factors determined in accordance with different standards.

16. The apparatus according to claim 10, wherein the program instructions are further configured to cause the apparatus to perform the following when executed by the at least one processor:
receive a request from a user device relating to emission values with respect to a specific conveying operation;
determine a starting location and a destination for the specific conveying operation;
read at least one stored emission factor, which is associated with the determined starting location and destination, for an entire route from the memory;
determine a numerical value for a transport unit which is associated with the emission factor;
calculate an emission value from the read emission factor and the determined numerical value for the transport unit; and
cause a transmission of the emission value to the user device.

17. The apparatus according to claim 16, wherein the program instructions are further configured to cause the apparatus to perform at least one of the following when executed by the at least one processor:
provide a web service which enables the request via the user device;
provide a web service for a shipment tracking by means of a user device which enables the request via the user device;
provide a web service for a shipment tracking by means of a user device which supports the request via the user device, wherein the specific conveying operation is indicated by means of a shipment identification which is input via the user device and wherein the starting location and the destination for the specific conveying operation and the numerical value for the transport unit for the specific conveying operation are determined by means of the shipment identification; and
provide a web service which enables the request via the user device and which enables an input of the starting location and the destination for the specific conveying operation and the numerical value for the transport unit for the specific conveying operation via the user device.

18. The apparatus according to claim 16, wherein the request comprises an indication of at least one desired standard, and wherein the reading of at least one emission factor from the store comprises reading at least one emission factor which is stored for the at least one desired standard.

19. An apparatus comprising at least one processor and at least one storage medium, with program instructions being stored in the at least one storage medium and with the program instructions being configured to cause the apparatus to perform the following when executed by the at least one processor:
receive a request from a user device relating to emission values with respect to a specific conveying operation;
determine a starting location and a destination for the specific conveying operation;
read at least one stored emission factor, which is associated with the determined starting location and destination, for an entire route from a memory, wherein the at least one emission factor is based on emission-relevant data relating to a multitude of carried out transport operations on route sections, from which the entire route can be composed, in a closed period of time;
determine a numerical value for a transport unit which is associated with the emission factor;
calculate an emission value from the read emission factor and the determined numerical value for the transport unit; and
cause a transmission of the emission value to the user device.

20. A non-transitory computer-readable storage medium storing program instructions that, when executed by a processor, cause an apparatus to perform the following:
receive emission-relevant data relating to a multitude of carried out transport operations on route sections from at least one server, the transport operations having been carried out within a closed time period of fixed length,
determine a respective emission factor for a plurality of possible entire routes which can be composed of the route sections, based on the emission-relevant data received, each emission factor representing an emission value for a transport operation on the entire route per transport unit, and
cause a storage of the determined emission factors for the plurality of possible entire routes in a memory with association with a starting location and a destination which define the respective entire route.
